(19) United States
(12) Patent Application Publication
(10) Pub. No.: US 2009/0324701 A1
(43) Pub. Date: Dec. 31, 2009

(54) COMPOSITIONS CONTAINING (S)-BETHANECHOL AND THEIR USE IN THE TREATMENT OF INSULIN RESISTANCE, TYPE 2 DIABETES, GLUCOSE INTOLERANCE AND RELATED DISORDERS

(75) Inventor: Mark Williams, Winnipeg (CA)

Correspondence Address: MERCHANT & GOULD PC, P.O. BOX 2903, MINNEAPOLIS, MN, 55402-0903, US

(73) Assignee: DIAMEDICA, INC., Manitoba (CA)

(21) Appl. No.: 12/161,577

(22) PCT Filed: Jan. 19, 2007
(86) PCT No.: PCT/CA07/00075

§ 371 Date: Jun. 3, 2009

Related U.S. Application Data
(60) Provisional application No. 60/758,450, filed on Jan. 11, 2006.

(51) Int. Cl. A61K 31/27; A61K 38/28; A61K 31/426; A61K 9/127; A61P 3/10

(52) U.S. Cl. 424/450; 514/478; 514/479; 514/4; 514/369

(57) ABSTRACT

The present invention provides pharmaceutical compositions comprising (S)-bethanechol or a pharmaceutically acceptable salt thereof, a pharmaceutically acceptable carrier and optionally at least one diabetes drug. The use of said composition in the treatment of insulin resistance, type 2 diabetes, impaired glucose tolerance and related disorders is also provided. The invention also provides for a kit comprising the pharmaceutical compositions and instructions for its use.

### FIELD OF INVENTION

The present invention relates to enantiomers of bethanechol, and in particular, the present invention relates to (S)-bethanechol and uses thereof for the treatment and prevention of insulin resistance and related disorders.

### BACKGROUND

Following a meal, hepatic parasympathetic nerves provide a permissive signal to the liver that regulates the ability of insulin to stimulate the release of a hormone, HISS, from the liver. HISS selectively stimulates glucose uptake and storage as glycogen in skeletal muscle and accounts for over one-half of the whole body glucose disposal that has previously been assumed to be a direct effect of insulin. Hepatic sympathetic nerves block the parasympathetic signal thus preventing the release of HISS and resulting in a 50% reduction in the glucose disposal effect of insulin. This condition is referred to as HISS-dependent insulin resistance (HDIR).

HISS action can be clinically diagnosed by determining the response to insulin in the fasted state and following re-feeding. The difference in the glucose disposal effect of an injection of insulin determined in the fed and fasted state represents the HISS-dependent component of insulin action. The glucose disposal produced in the fasted state is independent of HISS whereas the approximately doubled effect of insulin following a meal is due to both the HISS-dependent and HISS-independent component of insulin action with the difference between the two states being defined as the HISS-dependent component of insulin action.

HISS-dependent and HISS-independent insulin action can be most readily quantitated using the rapid insulin sensitivity test (RIST) which is a transient euglycemic clamp in response to a bolus administration of insulin. Normally insulin injection stimulates removal of glucose from the blood into storage sites with a resultant decrease in blood glucose level occurring. The RIST method uses variable glucose infusion rates to maintain the blood glucose level constant. The amount of glucose required to be administered in order to maintain the glycemic baseline is the index of insulin sensitivity and is referred to as the RIST index. The RIST index produced by this procedure consists of a HISS-dependent component and a HISS-independent component that can be readily differentiated by testing in the control fed state and then repeating the test after blockade of HISS release by any of a number of means including surgical denervation of the liver, blockade of hepatic muscarinic receptors, blockade of hepatic nitric oxide production, or blockade of hepatic cyclooxygenase. Eliminating HISS action by any of these procedures results in a reduction of the RIST index, in the fed state, of approximately 55%. That is, the glucose disposal effect that has been previously attributed to the direct action of insulin on a variety of tissues is actually mediated to a large extent by a hepatic insulin sensitizing process that has previously been unrecognized. This area has recently been reviewed (Lautt, 1999; Lautt, 2003). Blockade of HISS release results in HDIR. If HDIR is produced physiologically in response to fasting, these interventions do not produce any further decrease in insulin action.

HDIR is a normal and essential response to fasting. Insulin release occurs even in the fasted state and performs a number of growth regulating functions. Insulin is released in a pulsatile manner throughout the day with only approximately 50% of insulin release being regulated by food ingestion (Beyer et al., 1990). In the fasting state, it would be disadvantageous for insulin to cause a massive shifting of glucose from blood to skeletal muscle glycogen stores. The glucose disposal action in response to an injection of insulin decreases progressively to insignificance by 24 hours of fasting. This decrease in response to insulin represents a physiologically adjusted decrease in the HISS-dependent component as demonstrated by the observation that the HISS-independent (post-atropine or post-hepatic denervation) component of insulin action is similar in fed and 24-hour fasted rats.

In the immediate postprandial state, approximately 55% of the total glucose disposal effect of a bolus administration of insulin over a wide physiological range (5-100 mu/kg) is accounted for by HISS. By 18 hours of fasting, Sprague Dawley rats show HISS-dependent insulin action that accounts for only 26% of total insulin action (Lautt et al., 2001). The proportion of insulin action accounted for by HISS action remaining after 18 hours of fasting in cats is 35% (Xie & Lautt, 1995) and 25% in dogs (Moore et al., 2002). HISS action in rabbits accounts for approximately 44% of insulin action although the time since feeding was not stated (Porszasz et al., 2002). Fasting induces a 45% reduction in insulin action in mice (Latour & Chan, 2002). Preliminary results indicate that 62% of insulin action in the fed state is accounted for by HISS action in humans. This physiological regulation of HDIR is an appropriate response to fasting and, as such HDIR is a useful physiological state.

While HDIR is a useful physiological state in the fasted condition, failure to release HISS and the resultant HDIR in the fed state is suggested to account for the major metabolic disturbance seen in type 2 diabetes and many other conditions of insulin resistance. According to this model, post-meal nutrient processing normally results in approximately 80% of the glucose absorbed from a meal being stored in the large skeletal muscle mass of the body. Although HISS is released from the liver, it selectively stimulates glucose uptake into glycogen stores in skeletal muscle. Lack of HISS action results in a greatly impaired glucose disposal effect of insulin thus resulting in postprandial hyperglycemia. Additional insulin is released in response to the elevated glucose thus accounting for postprandial hyperinsulinemia in the type 2 diabetic. Insulin stimulates glucose uptake into adipose tissue and into the limited stores of the liver. When the glycogen stores in the liver are saturated, the remaining glucose is converted to lipid thus accounting for postprandial hyperlipidemia in the type 2 diabetic. The biochemical effects of hyperglycemia including the generation of free radicals has been suggested to account for the major non-metabolic pathologies common to diabetics including endothelial cell dysfunction, deposition of atherosclerotic plaques, blindness, renal failure, nerve damage, stroke, and hind limb amputation (Brownlee, 2001). HDIR has been shown to occur in chronic liver disease, fetal alcohol exposed adults, obesity, sucrose fed rats, hypertension, pregnancy and trauma.

The inventors propose that HDIR is the main cause for type 2 diabetes, impaired glucose tolerance, impaired fasting glucose, hyperinsulinemia, hyperlipidemia, obesity, postprandial hyperglycemia and other insulin resistant states. Bethanechol can increase insulin responsiveness by action at muscarinic receptors which are located in effector cells innervated by postganglionic parasympathetic nerves (U.S. Pat. No. 5,561,165).

### SUMMARY OF INVENTION

In one aspect, the present invention provides a pharmaceutical composition comprising an optically enriched (S)-bethanechol or pharmaceutically acceptable salt thereof, substantially free of its (R)-enantiomer and a pharmaceutically acceptable carrier. The (S)-bethanechol enantiomer surprisingly provides greater insulin responsiveness as compared to racemic bethanechol.

In an embodiment, the ratio of (S)-bethanechol to (R)-bethanechol contained in the pharmaceutical composition is 2:1 by weight.

In an embodiment, the ratio of (S)-bethanechol to (R)-bethanechol contained in the pharmaceutical composition is 3:1 by weight.

In an embodiment, the ratio of (S)-bethanechol to (R)-bethanechol contained in the pharmaceutical composition is 5:1 by weight.

In an embodiment, the ratio of (S)-bethanechol to (R)-bethanechol contained in the pharmaceutical composition is 10:1 by weight.

In an embodiment, the ratio of (S)-bethanechol to (R)-bethanechol contained in the pharmaceutical composition is 20:1 by weight.

In an embodiment, the optically enriched (S)-bethanechol is substantially free of its (R)-enantiomer.

In an embodiment, the optically enriched (S)-bethanechol comprises no more than about 25% w/w of the corresponding (R)-enantiomer.

In an embodiment, the optically enriched (S)-bethanechol comprises no more than about 10% w/w of the corresponding (R)-enantiomer.

In an embodiment, the optically enriched (S)-bethanechol comprises no more than about 5% w/w of the corresponding (R)-enantiomer

In an embodiment, the optically enriched (S)-bethanechol comprises no more than about 2% w/w of the corresponding (R)-enantiomer.

In a further aspect, the present invention provides a pharmaceutical composition comprising: (a) an optically enriched (S)-bethanechol or pharmaceutically acceptable salt thereof, substantially free of its (R)-enantiomer, (b) at least one diabetes drug and (c) a pharmaceutically acceptable carrier.

In an embodiment, the optically enriched (S)-bethanechol is substantially free of its (R)-enantiomer.

In an embodiment of the invention, the at least one diabetes drug may include, but is not limited a glutathione increasing compound, an antioxidant, an insulin or an insulin analogue, an α-adrenergic receptor antagonist, a β-adrenergic receptor antagonist, a non-selective adrenergic receptor antagonist, a sulphonylurea, a biguanide agent, a benzoic acid derivative, a α-glucosidase inhibitor, a thiazolidinedione, a phosphodiesterase inhibitor, a cholinesterase antagonist, or a GLP-1 analogue.

In an embodiment of the invention, the glutathione increasing compound is selected from a group consisting of: N-acetylcysteine, a cysteine ester, L-2-oxothiazolidine-4-carboxylate (OTC), gamma glutamylcysteine and its ethyl ester, glytathtione ethyl ester, glutathione isopropyl ester, α-lipoic acid, oxathiazolidine-4-carboxylic acid, cysteine, methionine, bucillamine and S-adenosylmethionine.

In an embodiment of the invention, the antioxidant is selected from a group consisting of: vitamin E, vitamin C, an isoflavone, zinc, selenium, ebselen, and a carotenoid.

In an embodiment of the invention, the insulin or insulin analogue is selected from the group consisting of: regular insulin, lente insulin, semilente insulin, ultralente insulin, NPH, Humalog® and Novolog®.

In an embodiment of the invention, the GLP-1 analogue is selected from a group consisting of: exanitide, DAC:GLP-1 (CJC-1131), liraglutide, ZP10, BIM51077, LY315902, and LY307161 (SR).

In an embodiment of the invention, the α-adrenergic receptor antagonist is selected from a group consisting of: prazosin, doxazocin, phenoxybenzamine, terazosin, phentolamine, rauwolscine, yohimine, tolazoline, tamsulosin, and terazosin.

In an embodiment of the invention, the β-adrenergic receptor antagonist is selected from a group consisting of: acebutolol, atenolol, betaxolol, bisoprolol, carteolol, esmolol, metoprolol, nadolol, penbutolol, pindolol, propranolol, timolol, dobutamine hydrochloride, alprenolol, bunolol, bupranolol, carazolol, epanolol, moloprolol, oxprenolol, pamatolol, talinolol, tiprenolol, tolamolol, and toliprolol.

In an embodiment of the invention, the non-selective adrenergic receptor antagonist is selected from a group consisting of: carvedilol and labetolol.

In an embodiment of the invention, the sulphonylurea is selected is from a group consisting of: tolazamide, tolubtuamide, chlorpropamide, acetohexamide, glyburide, glipizide, and glimepiride.

In an embodiment of the invention, the biguanide agent is metformin.

In an embodiment of the invention, the benzoic acid derivative is repaglinide.

In an embodiment of the invention, the α-glucosidase inhibitor is selected from a group consisting of: acarbose and miglitol.

In an embodiment of the invention, the thiazolidinedione is selected from a group consisting of: rosiglitazone, pioglitazone, and troglitazone.

In an embodiment of the invention, the phosphodiesterase inhibitor is selected from a group consisting of: anagrelide, tadalfil, dipyridamole, dyphylline, vardenafil, cilostazol, milrinone, theophylline, and caffeine.

In an embodiment of the invention, the cholinesterase antagonist is selected from a group consisting of: donepezil, tacrine, edrophonium, demecarium, pyridostigmine, zanapezil, phospholine, metrifonate, neostigmine, phenserine and galanthamine.

In an embodiment of the invention, the diabetes drug is N-acetylcysteine.

In an embodiment of the invention, the diabetes drug is α-lipoic acid.

In an embodiment of the invention, the pharmaceutical composition according to present invention further comprises a pharmaceutically acceptable liver targeting substance.

In an embodiment of the invention, the liver targeting substance is selected from a group consisting of: albumin, a liposome, and a bile salt.

In a further aspect, the present invention provides a use of the pharmaceutical composition according to the invention for treatment or prevention of a disorder selected from a group consisting of: type II diabetes, insulin resistance, impaired glucose intolerance, hyperglycemia, hyperlipidaemia, hyperinsulinemia, impaired glucose metabolism, obesity, diabetic retinopathy, diabetic nephropathy, glomerulosclerosis, syndrome X, hypertension, heart disease, cardiovascular disease, stroke, endothelial dysfunction, congestive heart failure, angina, peripheral arterial disease, chronic renal failure, and acute renal failure.

In a further aspect, the present invention provides a method of treating or inhibiting a disorder selected from a group consisting of: type II diabetes, insulin resistance, impaired glucose intolerance, hyperglycemia, hyperlipidaemia, hyperinsulinemia, impaired glucose metabolism, obesity, diabetic retinopathy, diabetic nephropathy, glomerulosclerosis, syndrome X, hypertension, heart disease, cardiovascular disease, stroke, endothelial dysfunction, congestive heart failure, angina, peripheral arterial disease, chronic renal failure, and acute renal failure, comprising administering a therapeutically effective amount of the pharmaceutical composition according to an embodiment of the invention.

In a further aspect, the present invention provides a method of treating or inhibiting a disorder selected from a group consisting of: type II diabetes, insulin resistance, impaired glucose intolerance, hyperglycemia, hyperlipidaemia, hyperinsulinemia, impaired glucose metabolism, obesity, diabetic retinopathy, diabetic nephropathy, glomerulosclerosis, syndrome X, hypertension, heart disease, cardiovascular disease, stroke, endothelial dysfunction, congestive heart failure, angina, peripheral arterial disease, chronic renal failure, and acute renal failure, comprising administering a therapeutically effective amount of (S)-bethanechol or pharmaceutically acceptable salt thereof and a therapeutically effective amount at least one diabetes drug.

In an embodiment, the amount of (S)-bethanechol is between 100 ug/day to 300 mg/day.

In an embodiment, the amount of (S)-bethanechol is between 1 mg/day to 300 mg/kg.

In an embodiment, the amount of (S)-bethanechol is between 3 mg/day to 300 mg/day.

In an embodiment, the amount of (S)-bethanechol is between 3 mg/day to 75 mg/day.

In an embodiment of the invention, the diabetes drug is N-acetylcysteine.

In an embodiment of the invention, the diabetes drug is α-lipoic acid.

In a further aspect, the present invention provides a kit comprising in combination: the pharmaceutical composition according to the invention and instructions for a dosage regimen for administration of said composition to ameliorate the symptoms a disorder selected from a group consisting of: type II diabetes, insulin resistance, impaired glucose intolerance, hyperglycemia, hyperlipidaemia, hyperinsulinemia, impaired glucose metabolism, obesity, diabetic retinopathy, diabetic nephropathy, glomerulosclerosis, syndrome X, hypertension, heart disease, cardiovascular disease, stroke, endothelial dysfunction, congestive heart failure, angina, chronic renal failure, and acute renal failure.

In an embodiment of the invention, the kit comprises a diabetes drug is selected from the group consisting of: a glutathione increasing compound, an antioxidant, an insulin or an insulin analogue, an α-adrenergic receptor antagonist, a β-adrenergic receptor antagonist, a non-selective adrenergic receptor antagonist, a sulphonylurea, a biguanide agent, a benzoic acid derivative, a α-glucosidase inhibitor, a thiazolidinedione, a phosphodiesterase inhibitor, a cholinesterase antagonist, and a GLP-1 analogue, and a pharmaceutical salt thereof, and the kit provides for a compartment for said diabetes drug and a second compartment for said (S)-bethanechol or pharmaceutically acceptable salt thereof, and said instructions provide for a dosage regimen for said diabetes drug and a second dosage regimen for said (S)-bethanechol or pharmaceutically acceptable salt thereof, wherein said dosage regimen is different from said second dosage regimen.

In an embodiment of the invention, the instructions include instructions to administer said pharmaceutical composition with a meal.

In an embodiment of the invention, the instructions include instructions to administer composition about 60 to 90 minutes before a meal.

In an embodiment of the invention, the diabetes drug is N-acetylcysteine.

In an embodiment of the invention, the diabetes drug is α-lipoic acid.

### BRIEF DESCRIPTION OF THE FIGURES

FIG. 1 is a line graph comparing the effect of racemic bethanechol, (S)-bethanechol and (R)-bethanechol for reversing denervation-induced HDIR.

FIG. 2 is a dose response curve for (S)-bethanechol and calculated percentage potentiation of denervated RISK index.

FIG. 3 is a logarithmic dose response curve for (S)-bethanechol and calculated percentage potentiation of denervated RISK index.

### DETAILED DESCRIPTION

While the present invention is not limited to a particular model or mechanism of action, it appears the parasympathetic response to feeding results in the release of acetylcholine which activates muscarinic receptors in the liver. This activation leads to increased production of nitric oxide which stimulates guanyl cyclase activity, resulting in increased levels of cyclic guanosine monophosphate which acts in stimulating the release of HISS. Feeding also results in elevated hepatic glutathione levels. Interruption of any component of this system can result in reduction or abolishment of the parasympathetic response to feeding. Accordingly, insulin resistance and related disorders may be the result of not only abnormal parasympathetic activity but also abnormal sympathetic activity.

In some instances, the parasympathetic function in response to feeding is impaired due to decreased acetylcholine production or release. In other instances, the parasympathetic function is impaired due to decreased nitric oxide production. The inventors have previously disclosed the use of cholinergic agonists (see for example, U.S. Pat. No. 5,561,165) such as bethanechol and nitric oxide donors.

Bethanechol is usually used clinically as a racemic mixture comprising both (R) and (S) enantiomers. The present inventors have previously disclosed the usefulness of racemic bethanechol for increasing insulin responsiveness. The present inventors have now determined that the (S)-enantiomer of bethanechol is provides increased insulin responsiveness as compared to racemic bethanechol and the (R)-enantiomer of bethanechol. While the invention is not limited to any particular theory of action, the inventors believe that the increased efficacy of the (S)-bethanechol over racemic bethanechol for increasing insulin responsive is related to differences in binding efficiency to muscarinic receptors. The inventors have determined that (S)-bethanechol has a higher binding efficiency for muscarinic receptors as compared to racemic bethanechol and (R)-bethanechol.

In one aspect, the present invention provides novel pharmaceutical compositions comprising an optically enriched (S)-bethanechol or pharmaceutically acceptable salt thereof. Methods for preparing (S)-enantiomer of bethanechol are set out in the Examples section of the present application. The pharmaceutical compositions according to the invention may contain some of the corresponding (R)-enantiomer. Preferably, the ratio of (S)-bethanechol to (R)-bethanechol in the pharmaceutical composition is 2:1 by weight; more preferably, the ratio of (S)-bethanechol to (R)-bethanechol in the pharmaceutical composition is 3:1 by weight; more preferably the ratio of (S)-bethanechol to (R)-bethanechol in the pharmaceutical composition is 5:1 by weight; more preferably the ratio of (S)-bethanechol to (R)-bethanechol in the pharmaceutical composition is 10:1 by weight; and more preferably the ratio of (S)-bethanechol to (R)-bethanechol in the pharmaceutical composition is 20:1 by weight.

In another aspect, the present invention provides novel pharmaceutical compositions comprising (S)-bethanechol or pharmaceutically acceptable salt thereof, substantially free of its (R)-enantiomer and a pharmaceutically acceptable carrier. Methods for preparing (S)-enantiomer of bethanechol are set out in the Examples section of the present application.

The term “substantially free of its corresponding (R)-enantiomer” means that the composition contains a greater proportion of the (S)-enantiomer as compared to the (R) enantiomer. Preferably, pharmaceutical compositions according to the invention contain no more than about 25% w/w of the corresponding (R)-enantiomer, more preferably no more than about 10% w/w of the corresponding (R)-enantiomer, more preferably no more than about 5% w/w of the corresponding (R)-enantiomer, more preferably no more than about 2% w/w of the corresponding (R)-enantiomer, and even more preferably no more than about 1% w/w of the corresponding (R)-enantiomer.

The term, “pharmaceutically acceptable salts”, refers to salts prepared from pharmaceutically acceptable non-toxic acids including inorganic acids and organic acids. Compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms. Preferably, the (S)-bethanechol is provides as its chloride salt form.

In a further aspect, the present invention provides a pharmaceutical composition comprising (a) (S)-bethanechol or pharmaceutically acceptable salt thereof, substantially free of its (R)-enantiomer, (b) at least one diabetes drug and (c) a pharmaceutically acceptable carrier.

As used herein, the term “diabetes drug” refers to any composition known in the art to be useful in the treatment or prevention of insulin resistance and diabetes. Examples of diabetes drugs which may be used to practice the invention include, but are not limited to:

an antioxidant such as vitamin E, vitamin C, an isoflavone, zinc, selenium, ebselen, a carotenoid;

an insulin or insulin analogue such as regular insulin, lente insulin, semilente insulin, ultralente insulin, NPH, Humalog®, or Novolog®;

an α-adrenergic receptor antagonist such as prazosin, doxazocin, phenoxybenzamine, terazosin, phentolamine, rauwolscine, yohimine, tolazoline, tamsulosin, or terazosin;

a β-adrenergic receptor antagonist such as acebutolol, atenolol, betaxolol, bisoprolol, carteolol, esmolol, metoprolol, nadolol, penbutolol, pindolol, propranolol, timolol, dobutamine hydrochloride, alprenolol, bunolol, bupranolol, carazolol, epanolol, moloprolol, oxprenolol, pamatolol, talinolol, tiprenolol, tolamolol, or toliprolol;

a non-selective adrenergic receptor antagonist such as carvedilol or labetolol;

a first generation sulphonylurea such as tolazamide, tolubtuamide, chlorpropamide, acetohexamide;

a second generation sulphonylurea such as glyburide, glipizide, and glimepiride;

a biguanide agent such as is metformin;

a benzoic acid derivative such as repaglinide;

a α-glucosidase inhibitor such as acarbose and miglitol;

a thiazolidinedione such as rosiglitazone, pioglitazone, or troglitazone;

a phosphodiesterase inhibitor such as anagrelide, tadalfil, dipyridamole, dyphylline, vardenafil, cilostazol, milrinone, theophylline, or caffeine;

a cholinesterase antagonist such as donepezil, tacrine, edrophonium, demecarium, pyridostigmine, zanapezil, phospholine, metrifonate, neostigmine, or galanthamine; and

a glutathione increasing compound such as N-acetylcysteine, a cysteine ester, L-2-oxothiazolidine-4-carboxylate (OTC), gamma glutamylcysteine and its ethyl ester, glytathtione ethyl ester, glutathione isopropyl ester, lipoic acid, cysteine, methionine, bucillamine or S-adenosylmethionine.

GLP and glucagon like peptide analogues, such as exanitide, DAC:GLP-1(CJC-1131), Liraglutide, ZP10, BIM51077, LY315902, LY307161 (SR).

In one preferred embodiment, a pharmaceutical composition comprises (S)-bethanechol in combination with N-acetylcysteine.

In another preferred embodiment, a pharmaceutical composition comprises (S)-bethanechol in combination with α-lipoic acid.

Pharmaceutical compositions of the present invention may further comprise pharmaceutically acceptably liver-targeting compounds. Inclusion of a liver-targeting compound allows the pharmaceutical compositions to be targeted to the liver of the patient, thereby eliminating deleterious systemic effects. The S-bethanechol and any other additional active ingredients can be conjugated to bile salts or albumin for preferential delivery to the liver. Alternatively, the S-bethanechol and any other additional active ingredients can be encapsulated within liposomes which are preferentially targeted to the liver. The pharmaceutical compositions of the present invention can be administered either in active form or as precursor which is metabolized by to the active form by enzymes in the liver. Where the pharmaceutical composition is targeted to the liver, the dosage may be reduced.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The pushfit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

Compositions may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example, subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a co-solvent system comprising benzyl alcohol, a nonpolar surfactant, a water-miscible organic polymer, and an aqueous phase. Naturally, the proportions of a co-solvent system may be varied considerably without destroying its solubility and toxicity characteristics. Furthermore, the identity of the co-solvent components may be varied.

Alternatively, other delivery systems for hydrophobic pharmaceutical compounds may be employed.

Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients.

Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable routes of administration may, for example, include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

The pharmaceutical compositions of the present invention may also include various other components which provide additional therapeutic benefit, act to affect the therapeutic action of the pharmaceutical composition, or act towards preventing any potential side effects which may be posed as a result of administration of the pharmaceutical composition. Exemplary pharmaceutically acceptable components or adjuncts which are employed in relevant circumstances include antioxidants, free radical scavenging agents, peptides, growth factors, antibiotics, bacteriostatic agents, immunosuppressives, anticoagulants, buffering agents, anti-inflammatory agents, anti-pyretics, time release binders, anesthetics, steroids, vitamins, and minerals.

Pharmaceutical compositions according to the invention can be used to treat or prevent insulin resistance and diabetes. Pharmaceutical compositions can also be used to treat or prevent other disorders related to insulin resistance such as impaired glucose intolerance, hyperglycemia, hyperlipidaemia, hyperinsulinemia, impaired glucose metabolism, obesity, diabetic retinopathy, diabetic nephropathy, glomerulosclerosis, syndrome X, hypertension, heart disease, cardiovascular disease, stroke, endothelial dysfunction, congestive heart failure, angina, chronic renal failure, acute renal failure and peripheral artery disease.

The terms “effective amount” or a “therapeutically effective amount” of a pharmacologically active agent refer to a nontoxic but sufficient amount of the drug or agent to provide a desired effect. In a combination therapy of the present invention, an “effective amount” of one component of the combination is the amount of that compound that is effective to provide the desired effect when used in combination with the other components of the combination. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

A therapeutic effective amount of any of the active agents encompassed by the invention will depend on number of factors which will be apparent to those skilled in the art and in light of the disclosure herein. In particular these factors include: the identity of the compounds to be administered, the formulation, the route of administration employed, the patient's gender, age, and weight, and the severity of the condition being treated and the presence of concurrent illness affecting the gastrointestinal tract, the hepatobiliary system and the renal system. Methods for determining dosage and toxicity are well known in the art with studies generally beginning in animals and then in humans if no significant animal toxicity is observed. The appropriateness of the dosage can be assessed by monitoring insulin resistance and liver function using the RIST protocol as set out in Lautt et al, 1998. Where the dose provided does not cause insulin resistance to decline to normal or tolerable levels, following at least three days of treatment, the dose can be increased. Patients should be monitored for signs of adverse drug reactions and toxicity, especially with regard to liver function.

For administration to mammals, and particularly humans, it is expected that the daily dosage level of (S)-bethanechol, will be between 100 ug and 300 mg and preferably between 1 mg and 300 mg for oral preparations. For parenteral or other systemic administration methods, the daily dosage level may be reduced. The daily dosage of the (S)-bethanechol is preferably between 250 μg and 10 mg for systemic administrations. In embodiments wherein the (S)-bethanechol is co-administered with at least one diabetes drug, a dosage of (S)-bethanechol may also be reduced.

Daily dosage of a diabetes drug will depend on the particular drug used. Where the diabetes drug is glizpide, a daily dosage will between 0.1 mg/kg and 10 mg/kg, and more preferably between 1 mg/kg and 5 mg/kg. Where the diabetes drug is acarbose, a daily dosage will be between 1 and 100 mg/kg, and preferably 10 mg/kg and 40 mg/kg. Where the diabetes drug is metformin, a daily dosage will be between 10 and 1000 mg/kg, and preferably 50 and 200 mg/kg. Where the diabetes drug is pioglitazone, a daily dosage will be between 0.1 and 10 mg/kg, and preferably between 0.5 mg/kg and 5 mg/kg. Where the diabetes drug is repaglinide, a daily dosage will be between 0.1 and 10 mg/kg, and preferably between 0.5 mg/kg and 5 mg/kg. Where the diabetes drug is N-acetylcysteine, a single dosage will be between 100 mg and 5 g, and preferably between 500 mg and 1 g daily. Where the diabetes drug is α-lipoic acid, a single dosage will be between 500 mg and 1 g daily.

A pharmaceutical composition may be administered to have it peak when blood glucose is high, such as after a meal, so as to allow glucose uptake at that time. Preferably, a pharmaceutical composition is administered 60 to 90 minutes prior to a meal.

In circumstances where it is desirable to administer a combination of (S)-bethanechol and at least one diabetes drug, the combination of drugs may be formulated into the same pill containing the (S)-bethanechol and the diabetes drug. Alternatively, a kit may be used comprising of multiple pills with an appropriate dose of (S)-bethanechol and the at least one diabetic drug, such as, but not limited to, a “blister pack”, including instructions or directions printed on or associated with the packaging.

Although the invention has been described with reference to illustrative embodiments, it is to be understood that the invention is not limited to these precise embodiments, and that various changes and modification are to be intended to be encompassed in the appended claims.

### EXAMPLES

### Example 1

### Synthesis of (R)-Bethanechol Chloride

All the preparations were carried out according to the procedures described in Micheli, C. et al, Il Farmaco—Edizione Scientifica 1983, 38(7), 514-20, plus some modifications.

The reactants (R)-(−)-1-Amino-2-propanol (1a) and (S)-(+)-1-Dimethylamino-2-propanol (2b) were commercially available from Aldrich company.

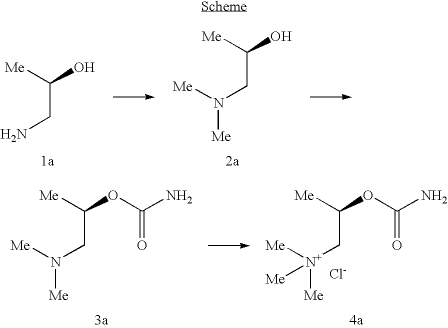

### Procedures

### Preparation of (R)-(−)-1-Dimethylamino-2-propanol (2a)

In a 100 mL round-bottom flask equipped with a magnetic stirring bar, and a refrigerant, (R)-(−)-1-Amino-2-propanol (3 mL, 37 mmol) was introduced under N₂ atmosphere, and cooled to 0° C. in ice bath. Formic acid (7 mL, 175 mmol) was added slowly and dropwise, followed with formaldehyde (5 mL, 67 mmol). The reaction mixture was heated at reflux for overnight, allowed to cool down at room temperature, and 6N HCl(aq) (25 mL) was added. The acidic mixture was washed with CH₂Cl₂ (3×20 mL), basified to pH 13 with a slow adjunction of 50% NaOH(aq) (40 mL), and extracted with CH₂Cl₂ (3×20 mL). The organic layer was dried over CaO, filtered, and allowed to warm up at 50° C. to evaporate the solvent at atmospheric pressure without a refrigerant. Crude yellowish oil 2a was obtained, and used in the next reaction without further purification.

### Preparation of (R)-(−)-2-Carbamyloxy-1-(N,N-dimethyl)-propylamine (3a)

In a 250 mL round-bottom flask equipped with a magnetic stirring bar, (R)-(−)-1-Diamino-2-propanol (2a) (5 mL, 40 mmol), and dry hexanes (80 mL) were added under N₂ atmosphere. The mixture was cooled to 0° C. in ice bath. Through a dropping funnel, a solution of chlorosulfonyl isocyanate (14 mL, 159 mmol) in dry hexanes (60 mL) was added slowly and dropwise under strong stirring. A colourless precipitate rapidly formed. The mixture was allowed to warm up to room temperature. The stirring was maintained for overnight. The reaction mixture was cooled to 0° C., and under strong stirring water was cautiously added dropwise until all the precipitate was dissolved. The aqueous layer was washed with CH₂Cl₂ (3×20 mL), basified to pH 13 with a cautious slow adjunction of 50% NaOH (aq) (50 mL), and extracted with CH₂Cl₂ (3×20 mL). The organic layer was dried over MgSO₄, filtered, and evaporated to dryness under reduced pressure to obtain colourless oil 3a that crystallized at room temperature.

### Preparation of (R)-(−)-2-Carbamyloxy-1-(N,N,N-trimethyl)-propylammonium chloride (4a)((R)-Bethanechol chloride)

To a solution of carbamyloxypropylamine 3a (40 mg, 0.27 mmol) in dry CH₂Cl₂ (2 ml), methyl iodide (85 μL, 1.36 mmol) was added. A colourless precipitate formed rapidly. The mixture was stirred at room temperature under N₂ atmosphere for overnight. The solvent was evaporated under reduced pressure, and the solid residue was dissolved in water, washed with CH₂Cl₂ (3×3 mL). The aqueous layer was concentrated to a minimum volume of water, charged on Chloride-exchange resin column, and eluted with bidistilled water. The elution was completed when a drop of the eluent did not precipitate with silver ions. The combined eluent was evaporated to dryness under reduced pressure, and the resulting crude crystals were purified by recrystallization from 2-propanol to give compound 4a as colourless crystals.

### Example 2

### Synthesis of (S)-Bethanechol Chloride

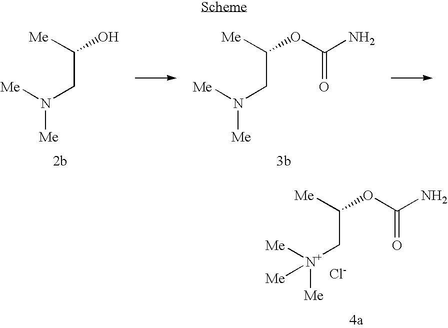

### Procedures

### Preparation of (S)-(+)-2-Carbamyloxy-1-(N,N-dimethyl)-propylamine (3b)

Compound 3b was prepared following the procedure described for the synthesis of 3a, but using commercially available (S)-(+)-1-Dimethylamino-2-propanol (Aldrich source).

### Preparation of (S)-(+)-2-Carbamyloxy-1-(N,N,N-trimethyl)-propylammonium chloride (4b) ((S)-Bethanechol chloride)

Compound 3b was reacted with methyl iodide to afford product 4b, following the procedure similar for the synthesis of 4a.

### Example 3

### Comparison of racemic bethanechol, R-bethanechol and (S)-bethanechol Binding to Muscarinic M₁ Receptors

### Radioligand Binding Muscarinic M₁ Binding Assay

The binding assay methodology was an adaptation of the methodology set out in Buckley N J, Bonner T I, Buckley C M and Brann M R (1989), Antagonist binding properties of five cloned muscarinic receptors expressed in CHO-K1 cells. Mol Pharmacol. 35(4): 469-476 and Luthin G R and Wolfe B B (1984), Comparison of [³H]pirenzepine and [³H]quinuclidinylbenzilate binding to muscarine cholinergic receptors in rat brain. J Pharmacol Exp Ther. 228(3):648-655.

The binding assay was performed under the following conditions:

Source: human recombinant CHO cells

### Ligand: 0.8 nM [³H] N-Methylscopolamine

### Vehicle: 1% DMSO

Incubation Time/Temp: 2 hours at 25° C.

### Incubation Buffer: 50 mM Tris-HCl, pH 7.4, 10 mM MgCl₂, 1 mM EDTA

### Non-specific Ligand: 1 μM Atropine

### K_{D}: 0.26 nM

Bₘₐₓ: 2 μmol/mg protein

### Specific Binding: 95%

Quantification Method: radioligand binding
Significance Criteria: ≧50% of max stimulation of inhibition

### Results

The binding affinity for muscarinic M₁ receptors was significantly higher for (S)-bethanechol as compared to R-bethanechol and racemic bethanechol. As shown in Table 1, (S)-bethanechol competitively inhibited [³H] N-methylscopolamine binding by 31% whereas R-bethanechol and racemic bethanechol did not compete with [³H] N-methylscopolamine for binding to muscarinic M₁ receptors.

TABLE 1
Competitive Binding of Racemic bethanechol, (R)-bethanechol
and (S)-bethanechol to M₁ receptors

| Compound (100 μm) | % Inhibition |
|---|---|
| racemic bethanechol | −4 |
| R-bethanechol | 0 |
| (S)-bethanechol | 31 |

### Example 4

### Comparison of racemic bethanechol, (R)-bethanechol and (S)-bethanechol Binding to Muscarinic M₁ Receptors

### Radioligand Binding Muscarinic M₃ Binding Assay

The binding assay methodology was an adaptation of the methodology set out in Buckley N J, Bonner T I, Buckley C M and Brann M R (1989), Antagonist binding properties of five cloned muscarinic receptors expressed in CHO-K1 cells. Mol Pharmacol. 35(4): 469-476 and Luthin G R and Wolfe B B (1984), Comparison of [³H]pirenzepine and [³H]quinuclidinylbenzilate binding to muscarine cholinergic receptors in rat brain. J Pharmacol Exp Ther. 228(3):648-655.

The binding assay was performed under the following conditions:

Source: human recombinant CHO cells

### Ligand: 0.8 nM [³H] N-Methylscopolamine

### Vehicle: 1% DMSO

Incubation Time/Temp: 2 hours at 25° C.

### Incubation Buffer: 50 mM Tris-HCl, pH 7.4, 10 mM MgCl₂, 1 mM EDTA

### Non-specific Ligand: 1 μM Atropine

### K_{D}: 0.75 nM

Bₘₐₓ: 534 pmol/mg protein

### Specific Binding: 95%

Quantification Method: radioligand binding
Significance Criteria: ≧50% of max stimulation of inhibition

### Results

The binding affinity for muscarinic M₃ receptors was significantly higher for (S)-bethanechol as compared to R-bethanechol and racemic bethanechol. As shown in Table 2, (S)-bethanechol competitively inhibited [³H] N-methylscopolamine binding by 42% whereas R-bethanechol and racemic bethanechol did not compete with [³H] N-methylscopolamine for binding to muscarinic M₃ receptors.

TABLE 2
Competitive Binding of Racemic bethanechol, (R)-bethanechol
and (S)-bethanechol to M₃ receptors

| Compound (100 μm) | % Inhibition |
|---|---|
| racemic bethanechol | −5 |
| (R)-bethanechol | −11 |
| (S)-bethanechol | 42 |

### Example 5

### Effect of Racemic Bethanechol on Insulin Sensitivity in Rats with Insulin Resistance Produced by Hepatic Denervation

The test subjects are male Spraque Dawely rats. The male rat is fasted for an 8 hour period and then re-fed for a 2 hour period. The rat is anesthetized with pentobarbital sodium (65 mg/kg) and is prepared surgically according to the standard animal preparation used to conduct a RIST test (Lautt et al., 1998). After surgery the rat stabilizes for 30 minutes.

Following the stabilization period and establishment of a baseline, a control RIST is carried out to show the amount of glucose needed to maintain euglycemia after a bolus administration of insulin (50 mU/kg i.v.). The response is within the normal range.

The animal undergoes surgery to cause insulin resistance. Surgical denervation of hepatic nerves reaching the liver along the hepatic artery is done. After an hour of recovery time a RIST is then carried out. The RIST index shows significant insulin resistance.

Racemic bethanechol is administered to the rat that has been surgically denervated with a total dose into the portal vein of (0.001 to 0.1) μg/kg beginning 10 minutes prior to the insulin administration. The resulting RIST index shows it restores insulin sensitivity.

### Example 6

### Effect of (R)-Bethanechol on Insulin Sensitivity in Rats with Insulin Resistance Produced by Hedatic Denervation

The test subjects are male Spraque Dawely rats. The male rat is fasted for an 8 hour period and then re-fed for a 2 hour period. The rat is anesthetized with pentobarbital sodium (65 mg/kg) and is prepared surgically according to the standard animal preparation used to conduct a RIST test (Lautt et al., 1998). After surgery the rat stabilizes for 30 minutes.

Following the stabilization period and establishment of a baseline, a control RIST is carried out to show the amount of glucose needed to maintain euglycemia after a bolus administration of insulin (50 mU/kg i.v.). The response is within the normal range.

The animal undergoes surgery to cause insulin resistance. Surgical denervation of hepatic nerves reaching the liver along the hepatic artery is done. After an hour of recovery time a RIST is then carried out. The RIST index shows significant insulin resistance.

(R)-bethanechol is administered to the rat that has been surgically denervated with a total dose into the portal vein of (0.001 to 0.1) μg/kg beginning 10 minutes prior to the insulin administration. The resulting RIST index shows it does not significantly restore insulin sensitivity.

### Example 7

### Effect of (S)-Bethanechol on Insulin Sensitivity in Rats with Insulin Resistance Produced by Hepatic Denervation

The test subjects are male Spraque Dawely rats. The male rat is fasted for an 8 hour period and then re-fed for a 2 hour period. The rat is anesthetized with pentobarbital sodium (65 mg/kg) and is prepared surgically according to the standard animal preparation used to conduct a RIST test (Lautt et al., 1998). After surgery the rat stabilizes for 30 minutes.

Following the stabilization period and establishment of a baseline, a control RIST is carried out to show the amount of glucose needed to maintain euglycemia after a bolus administration of insulin (50 mU/kg i.v.). The response is within the normal range.

The animal undergoes surgery to cause insulin resistance. Surgical denervation of hepatic nerves reaching the liver along the hepatic artery is done. After an hour of recovery time a RIST is then carried out. The RIST index shows significant insulin resistance.

(S)-bethanechol is administered to the rat that has been surgically denervated with a total dose into the portal vein of (0.001 to 0.1) μg/kg beginning 10 minutes prior to the insulin administration. The resulting RIST index shows it restores insulin sensitivity better than racemic bethanecol.

### Example 8

### Comparison of Effect of (S)-bethanechol, (R)-bethanechol and Racemic Bethanechol for Reversing HISS Dependent Insulin Resistance in Rats Caused by Anterior Hepatic Plexus Denervation

The test subjects were male Spraque Dawely rats (supplied by Charles River or The University of Manitoba) weighing between 222.0 to 375 g. The rats were separated into three test groups:

Group 1: Racemic Bethanechol

Group 2: (S)-Bethanechol

Group 3: (R)-Bethanechol

For each test group, the rats were fasted for an 8 hour period and then re-fed for a 2 hour period. The rats were anesthetized with pentobarbital sodium (65 mg/kg) and were prepared surgically according to the standard animal preparation used to conduct a control RIST test (Lautt et al., 1998). After surgery the rat stabilizes for 30 minutes. Following the stabilization period and establishment of a baseline, a control RIST was carried out to show the amount of glucose needed to maintain euglycemia after a bolus administration of insulin (50 mU/kg i.v.).

The animal underwent surgery to cause insulin resistance. Anterior plexus denervation and portal puncture was performed. After an hour of recovery time a post-denervation RIST was carried out. The inclusion criteria is a RIST index≧250 mg glucose/kg≦140 mg glucose/kg. For each test group, the test compound was administered at various doses μg/kg intraportal venous (0.5 mL bolus, rate of 0.05 mL/min plus 0.03 mL dead-space volume).

Following administration of the test compound, a post-administration RIST was carried out and the potentiation calculated as follows:

[(Reversal RIST Index−Blocked RIST Index)/Blocked RIST Index]×(100%)=% Potentiation.

If the potentiation was greater than 250% over the inhibited RIST, the animal was re-stabilized and a second post-administration RIST was performed to determine if the potentiation was sustained.

If the potentiation was less than 25%, the test compound was re-administered at a higher dose than the initial dose and a second post-administration RIST was performed.

Each test group was repeated using various doses of each test compound to establish a dose-response curve. The doses were determined based upon initial findings.

### Results

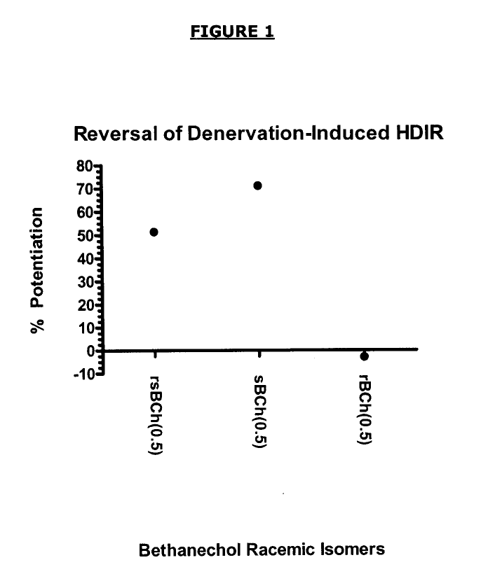

Test animals treated with (R)-bethanechol showed no significant reversal of denervation induced HDIR (see FIG. 1). In contrast, test animals treated with either racemic bethanechol or (S)-bethanechol showed substantial reversal of denervation induced HDIR (see FIG. 1). Treatment with (S)-bethanechol (ca. 72% potentiation) was found to be superior over treatment with racemic bethanechol (ca. 52% potentiation) for reversing denervation induced HDIR.

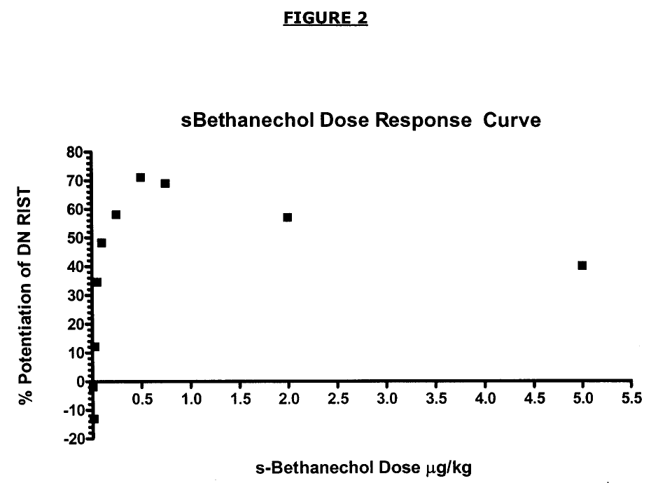

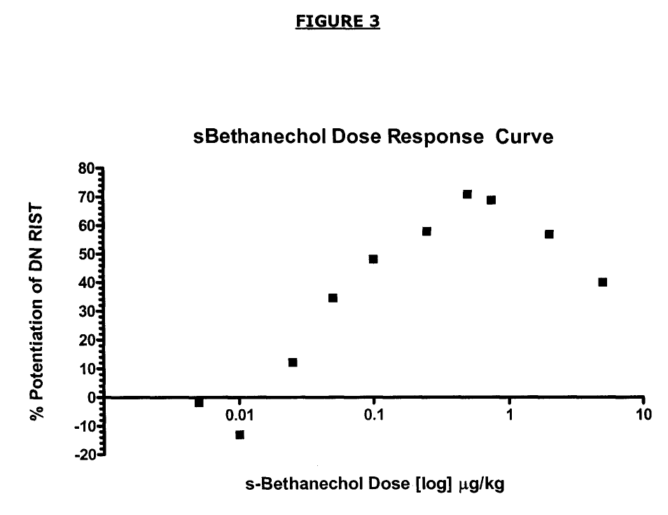

Dose response curves (FIGS. 2 and 3) were prepared for S-bethanchol. As shown in Table 3, the doses of (S)-bethanechol ranging between 0.025 to 5.000 μg/kg reversed denervation-induced HDIR, with doses between 0.250 and 2.000 μg/kg showing the greatest % potentiation.

TABLE 3
Comparison of (S)-bethanechol dose and % potentiation

| (S)-Bethanechol Dose | |
|---|---|
| (μg/kg) | % Potentiation |
| 0.005 | −1.81 |
| 0.010 | −13.01 |
| 0.025 | 12.24 |
| 0.050 | 34.66 |
| 0.100 | 48.29 |
| 0.250 | 58.16 |
| 0.500 | 71.19 |
| 0.750 | 69.08 |
| 2.000 | 57.21 |
| 5.000 | 40.27 |

### Example 9

### Comparison of Effect of (S)-bethanechol, (R)-bethanechol and Racemic Bethanechol for Increasing Hepatic Glutathione Levels in Denervated Rats

Hepatic glutathione (GSH) levels are determined for test animals from Example 8 which have a control RIST of ≧170 mg glucose/kg≦250 mg glucose mg/kg.

Following the post-denervation RIST analysis, a liver sample is taken from each test animal using cork borer #4 or #5 to remove a section of liver from the left lateral liver lobe. The top and bottom (1-2 mm) of the sample are trimmed and cut into 2 equal pieces. The samples are flash frozen on dry ice in tin foil, and are labeled with the experiment number and date. The samples are stored at −80 until GSH analysis is performed. Liver GSH levels are determined using the Bioxytech GSH420 kit.

### Results

Test animals treated with (S)-bethanechol show greater increases in hepatic GSH levels as are compared to test animals treated with (R)-bethanechol or racemic bethanechol.

### Example 10

### Comparison of Effect of (R)-bethanechol or (S)-bethanechol in Combination with N-Acetylcysteine (NAC) for Reversing HISS Dependent Insulin Resistance in Rats Caused by 35% Liquid Sucrose Model

The test subjects are male Spraque Dawely rats (to be supplied by Charles River or The University of Manitoba) weighing between 222.0 to 375 g. The rats are separated into four test groups:

Group 1: (R)-Bethanechol and NAC

Group 2: (S)-Bethanechol and NAC

Group 3: (R)-Bethanechol and saline control

Group 4: (S)-Bethanechol and saline control

For each test group, the rats are given ad libitum 35% sucrose water in addition to regular water for 63+/−7 days.

For each test group, the rats are fasted for an 8 hour period and then are re-fed for a 2 hour period. The rats are anesthetized with pentobarbital sodium (65 mg/kg) and are prepared surgically according to the standard animal preparation used to conduct a control RIST test (Lautt et al., 1998). After surgery the rat is stabilized for 30 minutes. Following the stabilization period and establishment of a baseline, a control RIST is carried out to show the amount of glucose needed to maintain euglycemia after a bolus administration of insulin (50 mU/kg i.v.) to obtain a pre-meal RIST1 value.

The rats are administered (S)- or (R)-bethanechol at an optimal dose of s-BCh as determined by previous experimental protocol. The (S)- or (R)-bethanechol is administered as a bolus volume of 0.5 mL plus 0.03 mL dead-space volume, at a rate of 0.05 mL/min. For test groups 1 and 2, the rats are also administered NAC at 200 mg/kg iv or an equivalent volume of saline as vehicle control for groups 3 and 4. The NAC or saline equivalent is administered as a bolus volume 1.0 mL, 0.1 mL/min iv.

A post-drug glycemic profile is performed with 5 minute samples taken out to 60 minutes post-initiation of the bethanechol infusion. A test meal (mixed liquid meal, 10 mL/kg), is then administered as an intra gastric infusion by a 10 mL/kg bolus dose, 1.0 mL/min. 0.1 mL test meal is added to account for catheter dead-space volume.

Blood glucose samples are taken every 5 minutes to profile the glycemic response to the test meal to obtain a minimum 90 minute profile.

If glycemia is stable at 90 minutes, a post meal RIST is performed to provide a RIST2 value. Otherwise, profiling is continued with 5 minute sampling until stable glycemia is achieved.

The MIS (percent potentiation of RIST2 over RIST1) is calculated as follows:

MIS=[(RIST2 INDEX mg/kg)−(RIST1 INDEX mg/kg)]/(RIST2 INDEX mg/kg)*100%

Following RIST analysis, a liver sample is taken from each test animal using cork borer #4 or #5 to remove a section of liver from the left lateral liver lobe for glutathione (GSH) testing. The top and bottom (1-2 mm) of the sample are trimmed and cut into 2 equal pieces. The samples are flash frozen on dry ice in tin foil, and are labeled with the experiment number and date. The samples are stored at −80 until GSH analysis is performed. Liver GSH levels are determined using the Bioxytech GSH420 kit.

RIST indices are compared using paired-T analysis within experiments, and group comparisons will be made with ANOVA.

### Results

Test animals having (S)-bethanechol and NAC combination therapy are shown by RIST analysis and hepatic GSH levels, to have improved reversal of insulin resistance induced by sucrose feeding as compared to test animals having (R)-bethanechol and NAC combination therapy. Test animals having (S)-bethanechol and NAC combination therapy are shown by RIST analysis and hepatic GSH levels, to have improved reversal of insulin resistance induced by sucrose feeding as compared to test animals having (S)-bethanechol therapy alone.

1-54. (canceled)

55. A pharmaceutical composition comprising optically enriched (S)-bethanechol, or a pharmaceutically acceptable salt thereof, and a pharmaceutically acceptable carrier, wherein the ratio of (S)-bethanechol to (R)-bethanechol in the pharmaceutical composition is at least 2:1 by weight.

56. The pharmaceutical composition according to claim 55, wherein the ratio of (S)-bethanechol to (R)-bethanechol is about 3:1, 5:1, 10:1, or 20:1.

57. A pharmaceutical composition comprising: (a) an optically enriched (S)-bethanechol or pharmaceutically acceptable salt thereof; (b) at least one diabetes drug; and (c) a pharmaceutically acceptable carrier.

58. The pharmaceutical composition according to claim 57, wherein the optically enriched (S)-bethanechol is substantially free of its (R)-enantiomer.

59. The pharmaceutical composition according to claim 57, wherein the optically enriched (S)-bethanechol comprises about 1% w/w to 25% w/w of the corresponding (R)-enantiomer.

60. The pharmaceutical composition according to claim 57, wherein the diabetes drug is selected from the group consisting of a glutathione increasing compound, an antioxidant, an insulin or an insulin analogue, an α-adrenergic receptor antagonist, a B-adrenergic receptor antagonist, a non-selective adrenergic receptor antagonist, a sulphonylurea, a biguanide agent, a benzoic acid derivative, a α-glucosidase inhibitor, a thiazolidinedione, a phosphodiesterase inhibitor, a cholinesterase antagonist, and a GLP-1 analogue.

61. The pharmaceutical composition according to claim 57, wherein the diabetes drug is N-acetylcysteine or α-lipoic acid.

62. The pharmaceutical composition according claim 57, further comprising a pharmaceutically acceptable liver targeting substance.

63. The pharmaceutical composition according to claim 62, wherein the liver targeting substance is selected from the group consisting of albumin, a liposome, and a bile salt.

64. A method of treating or inhibiting a disorder selected from the group consisting of type II diabetes, insulin resistance, impaired glucose intolerance, hyperglycemia, hyperlipidaemia, hyperinsulinemia, impaired glucose metabolism, obesity, diabetic retinopathy, diabetic nephropathy, glomerulosclerosis, syndrome X, hypertension, heart disease, cardiovascular disease, stroke, endothelial dysfunction, congestive heart failure, angina, peripheral arterial disease, chronic renal failure, and acute renal failure, comprising administering a therapeutically effective amount of the pharmaceutical composition according to claim 1 to a patient having said disorder.

65. A method of treating or inhibiting a disorder selected from a group consisting of: type II diabetes, insulin resistance, impaired glucose intolerance, hyperglycemia, hyperlipidaemia, hyperinsulinemia, impaired glucose metabolism, obesity, diabetic retinopathy, diabetic nephropathy, glomerulosclerosis, syndrome X, hypertension, heart disease, cardiovascular disease, stroke, endothelial dysfunction, congestive heart failure, angina, peripheral arterial disease, chronic renal failure, and acute renal failure, comprising administering a therapeutically effective amount of (S)-bethanechol, or pharmaceutically acceptable salt thereof, and a therapeutically effective amount at least one diabetes drug to a patient having said disorder.

66. The method according to claim 64, wherein the amount of (S)-bethanechol is about 100 μg/day to 300 mg/day.

67. The method according to claim 65, wherein the amount of (S)-bethanechol is between 100 μg/day to 300 mg/day

68. The method according to claim 65, wherein the diabetes drug is N-acetylcysteine or α-lipoic acid.

69. A kit comprising in combination: the pharmaceutical composition according to claim 55 and instructions for a dosage regimen for administration of said composition to ameliorate the symptoms of a disorder selected from a group consisting of: type II diabetes, insulin resistance, impaired glucose intolerance, hyperglycemia, hyperlipidaemia, hyperinsulinemia, impaired glucose metabolism, obesity, diabetic retinopathy, diabetic nephropathy, glomerulosclerosis, syndrome X, hypertension, heart disease, cardiovascular disease, stroke, endothelial dysfunction, congestive heart failure, angina, chronic renal failure, and acute renal failure.

70. A kit comprising in combination:
(i) a pharmaceutical composition comprising:
(a) an optically enriched (S)-bethanechol or pharmaceutically acceptable salt thereof;
(b) at least one diabetes drug, wherein the diabetes drug is selected from the group consisting of: a glutathione increasing compound, an antioxidant, an insulin or an insulin analogue, an α-adrenergic receptor antagonist, a β-adrenergic receptor antagonist, a non-selective adrenergic receptor antagonist, a sulphonylurea, a biguanide agent, a benzoic acid derivative, a α-glucosidase inhibitor, a thiazolidinedione, a phosphodiesterase inhibitor, a cholinesterase antagonist, and a GLP-1 analogue; and
(c) a pharmaceutically acceptable carrier;
(ii) a first compartment for said diabetes drug;
(iii) a second compartment for said (S)-bethanechol or pharmaceutically acceptable salt thereof; and
(iv) instructions providing for a dosage regimen for said diabetes drug and a second dosage regimen for said (S)-bethanechol or pharmaceutically acceptable salt thereof, wherein said dosage regimen is different from said second dosage regimen.

71. The kit of claim 70, wherein said instructions include instructions to administer said pharmaceutical composition with a meal.

72. The kit of claim 70, wherein said instructions include instructions to administer composition about 60 to 90 minutes before a meal.

73. The kit according to claims 72, wherein the diabetes drug is N-acetylcysteine or α-lipoic acid.
